(19) United States
(12) Patent Application Publication
(10) Pub. No.: US 2021/0121577 A1
(43) Pub. Date: Apr. 29, 2021

(54) FILM COATED TABLET FOR THE TREATMENT OF ACUTE PAIN

(71) Applicants: SANOFI-AVENTIS DEUTSCHLAND GMBH, Frankfurt am Main (DE); SANOFI-AVENTIS DE MEXICO SA DE CV, Mexico (MX)

(72) Inventors: Sandra SAUERLAND, Ingelheim am Rhein (DE); Thomas FUERST, Ingelheim am Rhein (DE); Alberto HEGEWISCH, Mexico (MX); Thomas WEISER, Ingelheim am Rhein (DE)

(21) Appl. No.: 17/064,482

(22) Filed: Oct. 6, 2020

Related U.S. Application Data
(63) Continuation of application No. 15/506,548, filed on Feb. 24, 2017, now abandoned.

(30) Foreign Application Priority Data
Aug. 28, 2014 (EP) 14002975.2

(51) Int. Cl. A61K 47/38; A61K 31/192; A61K 31/522; A61K 9/20

(52) U.S. Cl. CPC A61K 47/38; A61K 31/192; A61K 9/2095; A61K 9/2054; A61K 31/522

(57) ABSTRACT

The invention relates to a film coated tablet for the treatment of acute pain containing 400 mg of ibuprofen, 100 mg of caffeine, 50 to 100 mg of one or more disintegrating agents and to a process for the preparation of the film coated tablet according to the invention.

### CROSS REFERENCE TO RELATED APPLICATIONS

This application is a continuation of U.S. application Ser. No. 15/506,548, filed Feb. 24, 2017, which is a national stage application under 35 U.S.C. § 371 of International Application No. PCT/EP2015/066884, filed Jul. 23, 2015, which claims the priority of European Application No. 14002975.2 filed on Aug. 28, 2014.

### BACKGROUND OF THE INVENTION

### 1. Technical Area

The invention relates to a film coated tablet for the treatment of acute pain, containing 400 mg of ibuprofen, 100 mg of caffeine, 50 to 100 mg of one or more disintegrating agents.

### 2. Prior Art

Ibuprofen or (+/−) 2-(p-isobutylphenyl)-propionic acid of formula

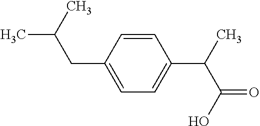

is known since long as an NSAID medication with analgesic and antipyretic activity.

Caffeine or 3,7-dihydro-1,3,7-trimethyl-1H-purine-2,6-dione of formula

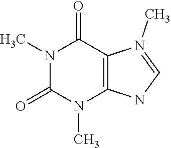

has long been used alone or together with other active substances for the treatment of acute pain.

U.S. Pat. No. 4,420,483 suggests the use of caffeine for accelerating the analgesic and anti-inflammatory activity of ibuprofen.

In European patent application EP 1 518 551 A1, solid pharmaceutical administration forms are suggested, which in addition to caffeine in uncoated form with a mean particle size of about 70 to 600μ contain a headache relieving agent, including ibuprofen.

The present invention was based on the aim of providing a pharmaceutical composition for oral administration which allows to treat acute pain of mild to severe intensity rapidly and alleviates pain for more than six hours.

Surprisingly, within the framework of extensive clinical trials it was shown that a film coated tablet containing 400 mg of ibuprofen, 100 mg of caffeine, 50 to 100 mg of one or more disintegrating agents is excellently suited for treating acute pain within a short time and with a long duration of action.

### BRIEF SUMMARY OF THE INVENTION

The present invention relates to a film coated tablet wherein the core of the tablet consists of 400 mg of ibuprofen, 100 mg of caffeine, 50 to 100 mg of one or more disintegrating agents and one or more further components selected from the group of carriers, flow regulating agents and lubricants, wherein the core of the tablet is obtainable by direct dry compression of all the components.

Another aspect of the present invention is a process for the manufacture of a film coated tablet in accordance with any one of the claims 1 to 8, wherein a mixture consisting of 400 mg of ibuprofen, 100 mg of caffeine, 50 to 100 mg of one or more disintegrating agents and one or more further components selected from the group of carriers, flow regulating agents and lubricants is dry compressed and coated with a coating.

A further aspect of the invention is a method for the treatment of acute pain, which method comprises administration of a film coated tablet wherein the core of the tablet consists of 400 mg of ibuprofen, 100 mg of caffeine, 50 to 100 mg of one or more disintegrating agents and one or more further components selected from the group of carriers, flow regulating agents and lubricants, wherein the core of the tablet is obtainable by direct dry compression of all the components to a person in need thereof.

### BRIEF DESCRIPTION OF THE DRAWINGS

FIGS. 1 and 2 show bar graphs of SPRID0-8 h and SPRID0-2 h, respectively. The fix dose combination (FDC) of ibuprofen and caffeine (“Ibup/Caff”) is 30-50% more effective than 400 mg ibuprofen alone. Values are shown as means+SEM adjusted for baseline pain intensity as measured on the 4-point verbal rating scale VRS.

FIG. 3 shows adjusted means for pain intensity difference over time.

FIG. 4 shows Kaplan-Meier estimates over time for time to perceptible pain relief.

FIG. 5 shows Kaplan-Meier estimates over time for time to meaningful pain relief.

### DETAILED DESCRIPTION OF THE INVENTION

The term “ibuprofen”, as used herein above and herein below, comprises the active substance 2-(p-isobutylphenyl)-propionic acid in any form, i.e., as a salt, as a free acid, as an enantiomer or enantiomer mixture; the racemate of the free acid is preferred.

The term “caffeine”, as used herein above and herein below, comprises natural and synthetic 3,7-dihydro-1,3,7-trimethyl-1H-purine-2,6-dione in any form as an amorphous powder or in the form of crystals with a certain particle size distributions.

The film coated tablet may be coated with conventional coating materials. The core of the tablet can contain conventional additives and excipients that are useful with film coated tablets, for example fillers, including water-soluble compressible carbohydrates, for example sucrose, mannitol, sorbitol, maltitol, xylitol, erythritol, lactose and mixtures thereof, conventional dry binders, including cellulose, cellulose derivatives, polyvinyl pyrrolidone, starch, modified starch and mixtures thereof; disintegrating agents such as microcrystalline cellulose, starch, sodium starch glycolate, cross-linked polyvinyl pyrrolidone, cross-linked carboxymethl cellulose or sodium croscarmellose; and lubricants, for example magnesium stearate, stearic acid, talc, vegetable oils and waxes. The core of the tablet can also contain pharmaceutically acceptable adjuvants, including for example preservatives, flavorings, acidifiers, antioxidants, lubricants, surfactants and colorings.

In one embodiment of the invention the core of the tablet comprises a directly compressed mixture of 100 mg caffeine and 400 mg ibuprofen in the form of a granulate, together with a carrier material, one or more disintegrating agents, a flow regulating agent and a lubricant, for example magnesium stearate or stearic acid.

The core of the tablet can be produced by dry direct pressing methods. In particular the dosage form can be produced by dry-mixing of caffeine, ibuprofen and the other excipients to form a mixture and compressing the mixture.

Preferred embodiments of the film coated tablet according to the invention are those in which

(a) the one or more disintegrating agents are selected from the group consisting of microcrystalline cellulose and croscarmellose; preferably, wherein a combination of microcrystalline cellulose and sodium croscarmellose is used as the disintegrating agent, in particular wherein the weight ratio of microcrystalline cellulose to sodium croscarmellose is 4-5 to 1;
(b) the carrier is selected from the group consisting of lactose, cellulose, saccharose, polyethylene glycol and polyethylene oxide (PEO); in particular, wherein the carrier is a mixture of different types of cellulose;
(c) the core of the tablet consist of 90 to 98% by weight of ibuprofen, caffeine and one or more disintegrating agents; in particular wherein the weight ratio between caffeine and one or more disintegrants is 1.0 to 0.1-0.9, preferably, wherein the weight ratio between caffeine and sodium croscarmellose is 5-10 to 1;
(d) it is used for the treatment of acute pain, preferably for the treatment of acute dental or jaw pain or for the treatment of acute headaches; in particular for the treatment of acute dental or jaw pain caused by dental extraction
(e) its pain-relieving effect begins within 15 to 180 minutes after administration in at least 15% of patients evaluated according to a Kaplan-Meyer analysis (e.g. Kaplan, E. L.; Meier, P. (1958). “Nonparametric estimation from incomplete observations”. J. Amer. Statist. Assn. 53 (282): 457-481. JSTOR 2281868);
(f) its pain-relieving effect lasts for at least 6 to 8 hours after administration in at least 60% of patients evaluated according to a Kaplan-Meyer analysis;
(g) it achieves a reduction of 5.9 on the numerical pain rating scale (NPRS) ranging from 0 to 10;
(h) it contains
10 to 50 mg of one or more carrier materials,
50 to 90 mg, of one or more disintegrating agents, in particular 62 to 88 mg of two disintegrating agents,
1 to 5 mg of one or more flow regulating agents, especially a colloidal silica, for example an Aerosil® product from the firm of Evonik Industries AG, Rodenbacher Chaussee 4, 63457 Hanau-Wolfgang, and
1 to 5 mg of one or more lubricants.

The following non-limiting examples will further illustrate the invention.

### Example 1

A film coated tablet is prepared containing:

| No. | Constituent | Quantity [mg] |
|---|---|---|
| 1 | Ibuprofen | 400 |
| 2 | Caffeine | 100 |
| 3 | Microcrystalline cellulose | 70.6 |
| 4 | Sodium croscarmellose | 15 |
| 5 | Cellulose | 24 |
| 6 | Colloidal silica | 2.6 |
| 7 | Magnesium stearate | 2.6 |
| 8 | Film Aqua Polish P | 15 |
| 9 | Water | 45 |

Components 1 to 7 are mixed together and pressed into a tablet. Subsequently the tablet is coated with constituents 8 and 9.

### Example 2

Clinical trials are performed in patients using the film coated tablets produced according to example 1 and using post-operative dental pain as a model for acute pain with the following study design:

In a single-center, randomized, two-stage, parallel-group double-blind study the efficacy and safety of the fixed combination of ibuprofen 400 mg and caffeine 100 mg (in the figures abbreviated as “Ibup/Caff”) was investigated in comparison with ibuprofen 400 mg, caffeine 100 mg and placebo in patients with post-operative dental pain.

| Study stage 1 | Study stage 2 | Number of patients |
|---|---|---|
| Ibuprofen + caffeine | Ibuprofen + caffeine | 210 |
| Ibuprofen | Ibuprofen | 210 |
| Caffeine | Ibuprofen + caffeine | 35 |
| Caffeine | Ibuprofen | 35 |
| Placebo | Ibuprofen + caffeine | 35 |
| Placebo | Ibuprofen | 35 |

Primary goal: Demonstration of the superior efficacy of the fixed combination of ibuprofen 400 mg and caffeine 100 mg compared with each of the individual active substances alone and compared with placebo for the treatment of post-operative dental pain over a period of 8 hours followed by a single dose of the medication (study stage 1).

Secondary goal: Evaluation of the efficacy and safety of multiple doses of the fixed combination compared with ibuprofen alone over a post-operative time period of 5 days (study stage 2).

Male and female patients between the ages of 18 and 55 years, scheduled for the extraction of 3 to 4 unsound wisdom teeth, with at least 2 extracted molars were recruited; the baseline of dental pain intensity must be at least moderate on a verbal evaluation scale and at least 5 on a numerical evaluation scale ranging from 0 to 10.

Following surgery, one film coated tablet every 6-8 hours for 5 days has been administered. Patients who received only placebo and caffeine were randomly switched to the ibuprofen or the ibuprofen/caffeine group after the first dose.

### Primary Endpoint

The time-weighted sum of pain relief (PAR) and the difference in pain intensity (PID) from 0 to 8 hours (SPRID0-8 H)

### Secondary Endpoint

The time-weighted sum of PAR and PID from 0 to 2 hours (SPRID0-2H)
Duration of pain relief
Time to significant pain relief

The pain intensity (PI) was evaluated in a diary before administration and at 0.25, 0.5, 0.75, 1, 1.5, 2, 3, 4, 5, 6, 7 and 8 hours after the first dose of the study medication using an 11-point numerical rating scale (NPRS) from 0=“no pain” to 10=“worst possible pain”.

The pain relief (PAR) beginning from pain onset was evaluated in a patient diary using a 5-point rating scale (VRS) (0=no pain relief; 1=a little pain relief; 2=some pain relief; 3=much pain relief; 4=complete pain relief) at the same time points as for the PI evaluation.

As soon as a patient needed an emergency medication or a second dose of the medication within less than 8 hours, PI and PAR were evaluated before the emergency medication or second dose was administered.

The time-weighted sum of pain relief (PAR) and the pain intensity difference (PID) relative to baseline between 0 and 8 hours is determined as follows:

SPRID0-8 h=(PID0.25+PAR0.25+PID0.5+PAR0.5+PID0.75+PAR0.75+PID1+PAR1)/4+(PID1.5+PAR1.5+PID2+PAR2)/2+PID3+PAR3+PID4+PAR4+PID5+PAR5+PID6+PAR6+PID7+PAR7+PID8+PAR8,

wherein the abbreviations PIDPID/PAR0.25/0.5/0.75/1/1.5/2/3/4/5/6/7/8 represent PID/PAR values at the times of 0.25, 0.5, 0.75, 1, 1.5, 2, 3, 4, 5, 6, 7 and 8 hours respectively
PID=PI at baseline−PI at the specific time point (here, higher PID values represent greater benefit for the patient).

Higher values of the SPRID0-8 h likewise indicate greater benefit for the patient.

A total of 70 patients were treated with placebo or caffeine, 279 patients with ibuprofen (1 patient did not participate in step II of the trial) and 282 patients with the combination.

Table I below gives the respective mean SPRID0-8 h value for the various treatments:

TABLE I
Adjusted Mean SPRID0-8 h value

| | | | | Ibuprofen + |
|---|---|---|---|---|
| Treatment | Placebo | Caffeine | Ibuprofen | caffeine |
| No. of patients | 70 | 70 | 209 | 213 |
| SPRID0-8 h | 10.554 | 15.824 | 40.165 | 52.291 |

The superiority of the combination over both individual therapies and placebo was demonstrated.

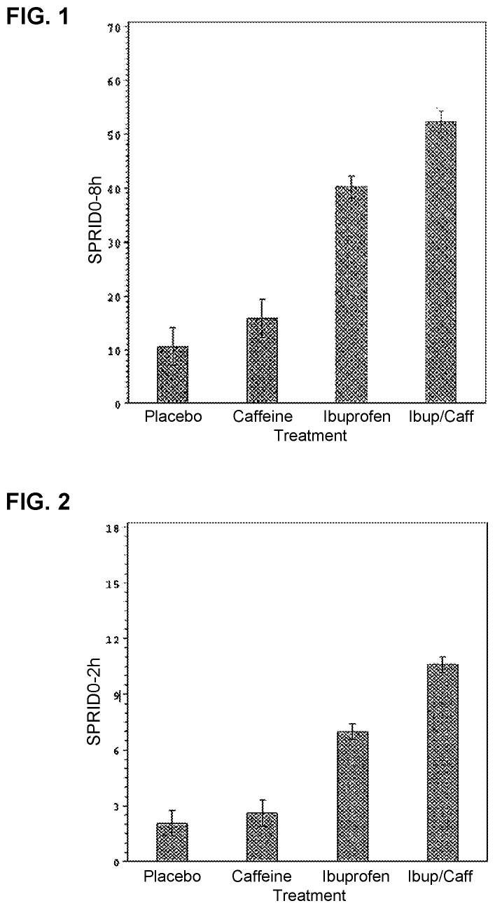

The combination of ibuprofen 400 mg and caffeine 100 mg demonstrated statistically significant superiority in terms of the primary endpoint SPRID0-8 h compared with both individual treatments and placebo (cp. FIG. 1).

The efficacy findings are in line with the results of a meta-analysis on caffeine as a co-analgesic (Derry C J, Derry S, Moore R A: Caffeine as an analgesic adjuvant for acute pain in adults (review), Cochrane Database Syst Rev (12), CD009281 (2014)). Interestingly, ibuprofen as analgesic shows a “ceiling effect”, i.e. single doses higher than 400 mg do not provide additional analgesia (Laska E M, Sunshine A, Marrero I, Olson N, Siegel C, McCormick N: The correlation between blood levels of ibuprofen and clinical analgesic response; Clin Pharmacol Ther 40 (1), 1-7 (1986); Kellstein D E, Lipton R B, Geetha R, Koronkiewicz K, Evans F T, Stewart W F, Wilkes K, Furey S A, Subramanian T, Cooper S A: Evaluation of a novel solubilized formulation of ibuprofen in the treatment of migraine headache: a randomized, double-blind, placebo-controlled, dose-ranging study; Cephalalgia 20, 233-243 (2000)). Thus, adding 100 mg caffeine to an ibuprofen dose of 400 mg provides more analgesic efficacy than expected for higher doses of ibuprofen alone.

The results of the primary endpoint were supported by the secondary endpoint SPRID0-2 h.

Table II below gives the respectively achieved mean SPRID0-2 h value for the various treatments (cp. FIG. 2):

TABLE II
Adjusted Mean SPRID0-2 h value

| | | | | Ibuprofen + |
|---|---|---|---|---|
| Treatment | Placebo | Caffeine | Ibuprofen | caffeine |
| No. of patients | 70 | 70 | 209 | 213 |
| SPRID0-2 h | 2.059 | 2.612 | 6.990 | 10.584 |

Table III below presents the median duration of action achieved according to Kaplan-Meyer analysis for the various treatments:

TABLE III
Median duration of action

| | | | | Ibuprofen + |
|---|---|---|---|---|
| Treatment | Placebo | Caffeine | Ibuprofen | caffeine |
| No. of patients | 70 | 70 | 209 | 213 |
| Duration of action | 1.6 | 2.1 | 7.1 | 7.3 |
| [hours] | | | | |

The combination according to the invention gave the longest duration of pain relief, followed by ibuprofen, caffeine and placebo.

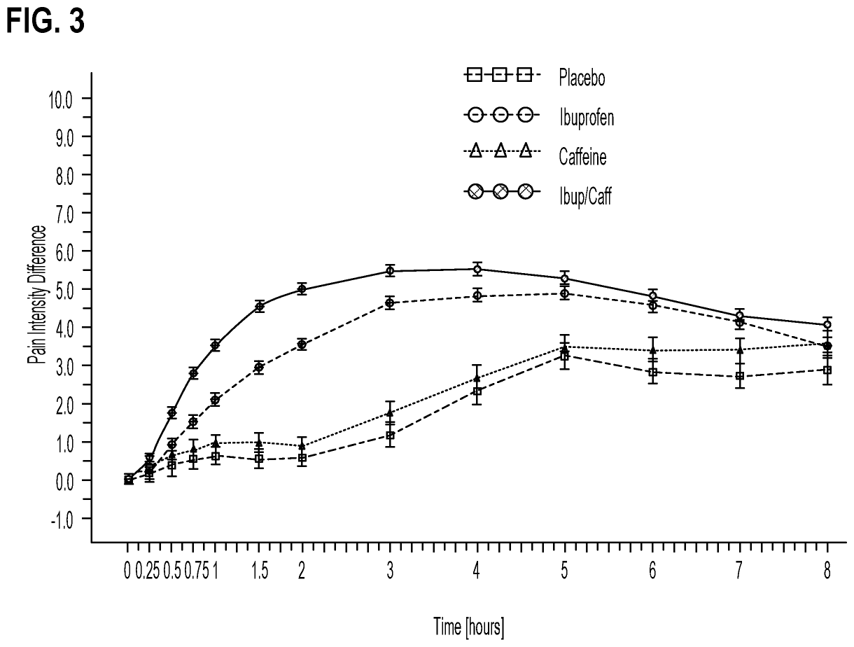

In addition, the analysis of pain intensity difference (as measured on the 0 to 10 numerical pain rating scale—NPRS) at individual time points corroborated the findings of the primary and secondary endpoint analyses. Treatment with ibuprofen/caffeine showed maintained analgesic efficacy with a fast onset, as demonstrated in the pairwise comparisons of adjusted mean pain intensities versus placebo, caffeine, and ibuprofen at individual time points. The comparison of ibuprofen/caffeine versus ibuprofen achieved statistical significance already after 0.5 h and up to 4 h of administration of trial medication. A reduction of ≥5.9 on the NPRS was only observed in the ibuprofen/caffeine arm and not in any of the other treatment arms at any time point. (cp. FIG. 3).

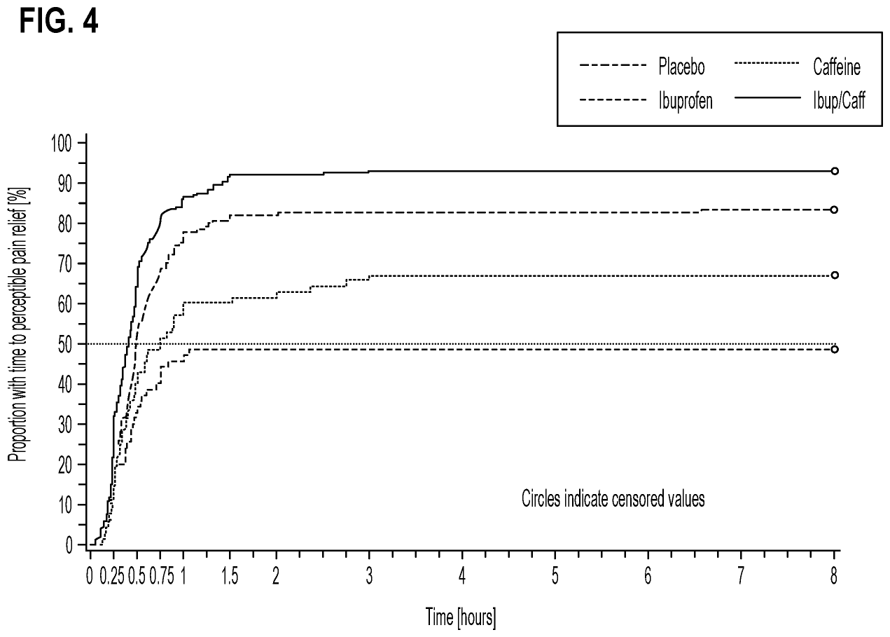

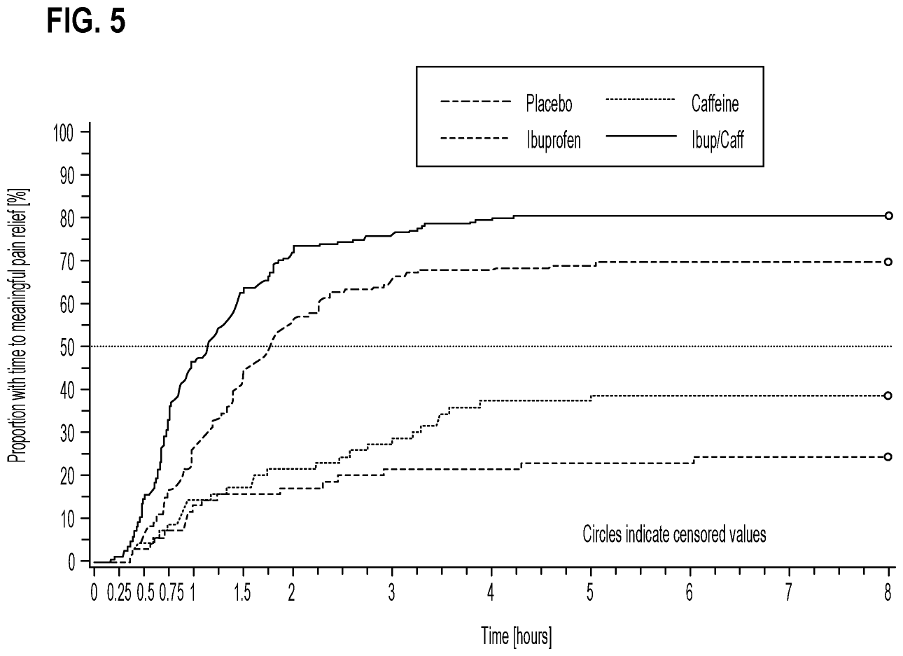

Table IV below gives the average time to significant pain relief according to Kaplan-Meyer analysis for the different treatments (cp. FIGS. 4 and 5):

TABLE IV
Median time to obtain relief

| | | | | Ibuprofen + |
|---|---|---|---|---|
| Treatment | Placebo | Caffeine | Ibuprofen | caffeine |
| No. of patients | 70 | 70 | 209 | 213 |
| Time [hours] | NC¹ | NC¹ | 1.78 | 1.13 |

¹NC = Not calculable (because more than half of the patients were without meaningful pain relief within 8 h)

The combination of ibuprofen 400 mg and caffeine 100 mg demonstrated significantly shorter times to meaningful pain relief compared with both individual treatments and placebo.

### Safety:

All treatments were safe and well-tolerated.

1. A film coated tablet wherein the core of the tablet consists essentially of:
400 mg of ibuprofen,
100 mg of caffeine,
62 to 88 mg of microcrystalline cellulose and sodium croscarmellose,
10 to 50 mg of cellulose,
1 to 5 mg of colloidal silica, and
1 to 5 mg of magnesium stearate;
wherein the core of the tablet comprises of 90 to 98% by weight of ibuprofen, caffeine, microcrystalline cellulose, and sodium croscarmellose that are dry compressed.

2. The tablet according to claim 1, wherein the cellulose is a mixture of different types of cellulose.

3. A process for the manufacture of the film coated tablet of claim 1, wherein a mixture consisting essentially of ibuprofen, caffeine, microcrystalline cellulose, sodium croscarmellose, cellulose, colloidal silica, and magnesium stearate is dry compressed and coated with a coating.

4. A method of treating acute pain, the method comprising administering the tablet according to claim 1.

5. The tablet according to claim 1, wherein microcrystalline cellulose is present in amount of 70.6 mg.

6. The tablet according to claim 1, wherein sodium croscarmellose is present in amount of 15 mg.

7. The tablet according to claim 5, wherein sodium croscarmellose is present in amount of 15 mg.

8. The tablet according to claim 1, wherein cellulose is present in amount of 24 mg.

9. The tablet according to claim 1, wherein colloidal silica is present in amount of 2.6 mg.

10. The tablet according to claim 1, wherein magnesium stearate is present in amount of 2.6 mg.

11. The tablet according to claim 1 comprising 70.6 mg of microcrystalline cellulose, 15 mg of sodium croscarmellose, 24 mg of cellulose, 2.6 mg of colloidal silica, and 2.6 mg of magnesium stearate.
